(19) United States
(12) Patent Application Publication
(10) Pub. No.: US 2007/0196350 A1
(43) Pub. Date: Aug. 23, 2007

(54) Compositions and Methods for Effecting Controlled Posterior Vitreous Detachment

(76) Inventor: Stephen P. Bartels, Pittsford, NY (US)

Correspondence Address: Bausch & Lomb Incorporated, One Bausch & Lomb Place, Rochester, NY, 14604-2701, US

(21) Appl. No.: 11/671,672

(22) Filed: Feb. 6, 2007

Related U.S. Application Data
(60) Provisional application No. 60/775,738, filed on Feb. 22, 2006.

(51) Int. Cl. A61K 38/48; A61K 38/44; A61K 31/7076; A61K 31/192; A61K 31/573; A61K 31/616; A61K 31/612; A61K 31/541; A61K 31/195

(52) U.S. Cl. 424/94.4; 424/94.64; 424/489; 514/171; 514/165; 514/569; 514/570; 514/406; 514/561; 514/47; 514/227.5; 514/731; 514/563

(57) ABSTRACT

A composition comprises plasmin or an enzymatically equivalent derivative thereof and at least an anti-inflammatory medicament. The composition can be used to effect or induce a controlled posterior vitreous detachment (“PVD”) to prevent, treat, or ameliorate a potential complication of a pathological ocular condition. Such a composition can be administered intravitreally.

This application claims the benefit of Provisional Patent Application No. 60/775,738 filed Feb. 22, 2006, which is incorporated by reference herein.

### BACKGROUND OF THE INVENTION

The present invention relates to compositions and methods for effecting a controlled posterior vitreous detachment (“PVD”). In particular, the present invention relates to such compositions and methods of treatment that treat, reduce, or ameliorate at least a cause or effect of a condition that prompts a need of such a controlled PVD. More particularly, the present invention relates to such compositions comprising plasmin or an equivalent thereof and an anti-inflammatory medicament and to methods for effecting such a controlled PVD using such compositions.

The vitreous is a clear mass that fills the posterior cavity of the eye between the lens and the retina. It is composed mostly of water and contains various amounts of salts, soluble proteins, glycoproteins, and glycosaminoglycans (mostly hyaluronic acid). The vitreous is attached at its posterior face to the retina along the structure known as the internal limiting membrane. This site of attachment of the vitreous and the retina is termed the vitreoretinal junction and consists of a layer of basement membrane proximal to the retina and a layer of collagen fibrils proximal to the vitreous.

PVD is the separation of the vitreous from the retina. Degenerative changes in the vitreous are a precursor to PVD. Degeneration of the vitreous is part of the normal aging process but also may be induced by pathological conditions such as diabetes, Eales' disease (which manifests as extraretinal hemorrhages), and uveitis (see, e.g., “Retinal Detachment” at http://www.emedicine.com/emerg/topic504.html). Degeneration of the vitreous results in its shrinkage and pulling away from the retina. Because the vitreous is attached to the retina, the receding vitreous can cause a retinal tear if it pulls hard on the retina—a process called traction, with subsequent detachment of the retina. In addition, as traction is exerted on the retina, inflammation can result in the tissues surrounding the points where the vitreous is still attached to the retina.

Certain pathological conditions of the eye are accompanied by the formation of new (abnormal) membranes in some cases with vessels, i.e. fibrovascular membranes, on the surface of the retina—namely proliferative diseases. With a naturally occurring PVD, traction is placed on these membranes and in those with new vessels there can be rupture and bleeding, which can result in fluid accumulation within and/or under the retina and hemorrhage interior to, within and/or under the retina. Proliferative retinal diseases thus are accompanied by retinal traction and both a high probability of retinal detachment, retinal edema as well as complications from bleeding resulting from the rupture of the newly formed blood vessels. Thus, it is beneficial to induce a controlled PVD before damage to the retina occurs because of uncontrolled detachment. Further, it is thought that attachments between the vitreous and the retina can serve as a scaffold for the abnormal growth of new fibrovascular membranes from the retina and into the vitreous of patients suffering from proliferative back-of-the-eye disorders. Thus, creation of a controlled PVD may avoid or inhibit such growth of fibrovascular membranes into the vitreous.

Verstraeten et al. (Arch. Ophthalmol., Vol.11, 849-854 (1993)) proposed the use of plasmin to produce a cleavage at the vitreoretinal interface. Plasmin hydrolyzes glycoproteins, including laminin and fibronectin, which are found at the vitreoretinal junction. Plasmin treatment was performed with or without subsequent vitrectomy on rabbit eyes. The authors noted that eyes treated with plasmin showed some areas of PVD, but only after vitrectomy was the vitreous substantially detached. The authors concluded that plasmin treatment may be useful as a biochemical adjunct to mechanical vitrectomy.

Plasmin has been proposed for inducing controlled PVD to prevent, stop, or reduce the progression of retinal detachment. U.S. patent application Ser. No. 11/126,625 having the common assignee teaches that creation of a controlled PVD is thought to inhibit the progression of nonproliferative diabetic retinopathy. That application and the references disclosed therein are incorporated herein by reference. However, administration of plasmin alone into a patient may not address the cause or effect of the original condition that prompts the need of effecting the controlled PVD or accelerate the return of the diseased eye to normal health.

Therefore, there is a need to provide improved compositions and methods for effecting a controlled PVD to treat, reduce, or ameliorate a cause or effect of a condition that initiates a need thereof. It is also very desirable to provide such compositions and methods, which employ non-toxic levels of active ingredients, to effect such a controlled PVD.

### SUMMARY OF THE INVENTION

In general, the present invention provides compositions comprising plasmin or an enzymatically equivalent derivative thereof and one or more anti-inflammatory medicaments.

In one aspect, a composition of the present invention can induce a controlled PVD to prevent, stop, reduce, or ameliorate at least an effect or complication of an ocular condition that initiates the need of such a controlled PVD.

In another aspect, a composition of the present invention also can effect a treatment, stoppage, reduction, or amelioration of at least a potential cause of an ocular condition that initiates the need of such a controlled PVD.

In still another aspect, the anti-inflammatory medicament has low solubility (defined below) in the vitreous.

In still another aspect, the present invention also provides methods for making and using such compositions.

In yet another aspect, a composition of the present invention comprises plasmin or an enzymatically equivalent derivative thereof and at least an anti-inflammatory medicament selected from the group consisting of corticosteroids, non-steroid anti-inflammatory drugs (“NSAIDs”), peroxisome proliferator-activated receptor-γ (“PPARγ”) ligands, combinations thereof, and mixtures thereof.

In still another aspect, the present invention provides a method for effecting a controlled PVD. The method comprises administering to or into an eye of a subject in need of said controlled PVD a therapeutically effective amount of a composition that comprises plasmin or an enzymatically equivalent derivative thereof and at least an anti-inflammatory medicament.

In a further aspect, the present invention provides a use of plasmin or an enzymatically equivalent derivative thereof and at least an anti-inflammatory medicament for the manufacture of compositions usable for effecting a controlled PVD in a subject in need thereof.

Other features and advantages of the present invention will become apparent from the following detailed description and claims.

### DETAILED DESCRIPTION OF THE INVENTION

In general, the present invention provides compositions comprising plasmin or an enzymatically equivalent derivative thereof and at least an anti-inflammatory medicament and methods of making and/or using such compositions.

In one aspect a composition of the present invention can induce a controlled PVD to prevent, stop, reduce, or ameliorate at least an effect or complication of an ocular condition that initiates the need of such a controlled PVD. In one embodiment, said at least an effect or complication is an inflammation of an ocular tissue. In another embodiment, said at least an effect or complication is an inflammation of an ocular tissue adjacent to the point where the vitreous is still attached to the retina prior to such a controlled PVD.

In another aspect, a composition of the present invention also can effect a treatment, stoppage, reduction, or amelioration of at least a potential cause of an ocular condition that initiates the need of such a controlled PVD. In one embodiment, said at least a potential cause is an inflammation of an ocular tissue (e.g., vitritis, uveitis, ocular toxoplasmosis, toxocariasis, pars planitis, macular edema or ocular contusion), extraretinal hemorrhages (e.g., as a result of Eales' disease), a vasoproliferative ocular disorder (e.g., retinal neovascularization, diabetic retinopathy, wet macular degeneration or choroidal neovascularization (“CNV”)), diabetic macular edema (“DME”) or an ocular injury or disorder (e.g., retina tear, retina detachment, epiretinal membrane, macular pucker or macular hole).

As used herein, the term “enzymatically equivalent derivative” of plasmin means an enzyme that is derived from plasmin and has a proteolytic function similar to that of plasmin. A derivative of plasmin can be a fragment or a variant of plasmin that has a proteolytic function similar to that of plasmin. A derivative of plasmin can be microplasmin comprising the enzymatic domain of plasmin and a short amino acid sequence (e.g., comprising about 20-40 amino acid residues) at the amino terminus of the enzymatic domain, miniplasmin comprising the enzymatic domain attached to the kringle-5 domain of plasmin, or other truncated forms of plasmin that comprise the enzymatic domain and one or more kringle domains of plasmin having retained lysine-binding property. A variant of plasmin can be generated from a molecule of plasmin by deleting, substituting, or adding one or more amino acid residues. Such substitution can be, for example, a conservative substitution. Enzymatically active microplasmin and miniplasmin are obtained from microplasminogen and miniplasminogen precursors by cleavage of the peptide bond at Arg⁵⁶¹-Val⁵⁶², wherein the amino acid residue numbers correspond to those of human Glu-plasminogen, which has 791 amino acid residues. Microplasmin is disclosed in, for example, U.S. Pat. No. 4,774,087; and miniplasmin is disclosed in, for example, U.S. Patent Application Publications 2005/0118158 and 2005/0124036. The contents of these documents are incorporated herein by reference.

In one aspect, a truncated plasmin comprises the enzymatic domain of plasmin attached at its amino terminus to kringle-1, kringle-2, kringle-3, kringle-4, or kringle-5 domain of plasmin, or combinations thereof. In one embodiment, two or more kringle domains are attached in any order to the amino terminus of the enzymatic domain. A kringle domain of plasmin is characterized by a triple-loop conformation and comprises about 75-85 amino acid residues with three disulfide bridges.

In another aspect, the enzymatically equivalent derivative of plasmin is microplasmin, miniplasmin, or a truncated plasmin. In one embodiment, the truncated plasmin comprises the kringle-1 domain of plasmin attached to the enzymatic domain of plasmin at the amino terminus of the enzymatic domain. In another embodiment, the kringle-1 domain in the truncated plasmin is substituted with the kringle-2, kringle-3, kringle-4, or kringle-5 domain. In still another embodiment, the truncated plasmin comprises two or more, but fewer than five, kringle domains attached in any order to the amino terminus of the enzymatic domain.

The term “combination” encompasses, but is not limited to, two or more molecules or fragments of molecules attached, attracted, held, or adhered together by bonds, attraction, or interaction (including, but not limited to, hydrogen bonding, ionic bonding, physical (such as by van der Waals force) or chemical adsorption, covalent bonding, or organometallic interaction), two interpenetrating molecules, or a complex comprising two or more molecules by, e.g., bonding or conformational interaction.

Plasmin is a serine protease that mediates the fribrinolytic process and modulates the extracellular matrix. It hydrolyzes a variety of glycoproteins, including laminin and fibronectin, both of which are present at the vitreoretinal interface and are thought to play a key role in vitreoretinal attachment. Plasmin does not degrade type-IV collagen, a major component of basement membranes and the inner limiting membrane (“ILM”) (see, e.g., A. Gandorfer et al., Investigative Ophthalmology & Visual Science, Vol. 45, No. 2, 641-47 (2004)). Enzymatically equivalent derivatives of plasmin, having the enzymatic domain of plasmin, can thus hydrolyze the same types of polypeptide substrates. Therefore, although the applicant does not wish to be bound by any particular theory, he believes that plasmin and its enzymatically equivalent derivatives hold promise to induce a controlled PVD without damaging the ILM and the retina. Therefore, in one aspect of the present invention, plasmin and/or an enzymatically equivalent derivative thereof can be administered intravitreally to induce a controlled PVD by hydrolyzing selected proteins, including laminin and fibronectin, at the vitreoretinal interface.

Besides the normal aging process, many pathological conditions also can initiate the onset of or accelerate uncontrolled PVD. For example, in the first and second decades of life, retinitis, nondiabetic retinal vascular disorders, and ocular contusion are common conditions that can initiate uncontrolled PVD. Beginning in the third decade, proliferative diabetic retinopathy and its associated complications are known to cause uncontrolled PVD. In addition, many intraocular inflammatory or infectious diseases of various etiologies result in opacification and/or liquefaction of the vitreous, and eventually uncontrolled PVD, leading to retinal tear. Non-limiting examples of these diseased conditions are ocular toxoplasmosis, toxocariasis, and pars planitis. Although other pathological ocular conditions (such as the proliferative ocular disorders) exhibiting uncontrolled PVD do not appear to be themselves inflammatory conditions, they can elicit inflammatory response in tissues adjacent to the point of uncontrolled PVD. The inflammatory response can lead to angiogenesis and thus the proliferative nature of these disorders.

It has been known that angiogenesis and chronic inflammation are codependent (see, e.g., J. R. Jackson et al., The FASEB J., Vol. 11, 457-65 (1997)). Chronic inflammation involves proliferation, migration, and recruitment of tissue and inflammatory cells, which can be extremely damaging to normal tissue. In all cases, the proliferating tissue contains an abundance of inflammatory cells, angiogenic blood vessels, and derived inflammatory mediators. There are also zones of relative hypoxia where tissue proliferation has outstripped blood vessel growth, which induces further capillary development. Macrophages, for example, are induced to release large quantities of angiogenic factors under hypoxic conditions. Inflammatory mediators can also, either directly or indirectly, promote angiogenesis. Angiogenesis, in turn, contributes to inflammatory pathology. New blood vessels can maintain the chronic inflammatory state by transporting inflammatory cells to the site of inflammation and supplying nutrients and oxygen to the proliferating inflamed tissue. The increased endothelial surface area also creates an enormous capacity for the production of cytokines, adhesion molecules, and other inflammatory stimuli.

Many types of cells are capable of producing angiogenic factors. Of all these types of cells, the inflammatory monocyte/macrophage type can be found at most sites where angiogenesis is occurring in an abnormal environment, including wounds and diseased tissues. In general, macrophages can induce angiogenesis via different mechanisms. First, macrophages can secrete factors (e.g., basic fibroblast growth factor (“bFGF”), vascular endothelial growth factor (“VEGF”), transforming growth factors α and β, (“TGF-α” and “TGF-β”), and platelet-derived growth factor (“PDGF”)) that can directly induce new blood vessel growth, or indirectly stimulate other cell types to secrete additional or higher levels of angiogenic factors. Macrophages can be activated to be angiogenic under conditions of hypoxia. Second, macrophages are capable of secreting factors that may degrade connective tissue matrix, which is critical in endothelial cell biology. Thus, it is entirely reasonable to view many proliferative ocular diseases as having genesis in inflammation.

For example, proliferative diabetic retinopathy begins with the poor circulation of blood in retinal blood vessels. As the cells of these vessels become starved because of poor supply of nutrients and oxygen, they become damaged and die, and the vessels are closed off. This process leads to ischemia and hypoxia of the retina, which are the first steps in the development of proliferative diabetic retinopathy (“PDR”). The condition of ischemia and hypoxia can stimulate the recruitment of inflammatory cells, including macrophages, to the site of injury, and, later, the growth of new blood vessels, as the tissue attempts to regain homeostasis. Importantly, the new blood vessels often grow on the surface of the retina and at the optic nerve. These new blood vessels are fragile, have high permeability, and are prone to bleed. As these blood vessels grow, they can exert traction on the retina, pulling on the retina and even leading to retinal detachment. Furthermore, it is possible that several factors or enzymes that facilitate the growth of these new blood vessels (for example, by degrading supporting tissues of the retina) also initiate the onset of uncontrolled, pathological PVD through their proteolytic action.

Therefore, recognizing the intricate relationship between uncontrolled, pathological PVD and potential root causes of many disease conditions that bring about such an uncontrolled, pathological PVD, the applicant provides compositions and methods for effecting or inducing a controlled PVD to prevent, stop, or reduce the potentially damaging complications that would result if the uncontrolled, pathological PVD is allowed to progress. Although the applicant does not wish to be bound by any particular theory, a composition of the present invention provides plasmin or an enzymatically equivalent derivative thereof (as disclosed above) to effect or induce a controlled separation of the vitreous from the inner limiting membrane, and at least an anti-inflammatory medicament to treat, reduce, or ameliorate at least a potential cause and/or to prevent, stop, reduce, or ameliorate at least an effect of an ocular condition that initiates the need of such a controlled separation.

Thus, in one aspect, the compositions and methods of the present invention are useful in preventing, treat, stop, reduce, or ameliorate an ocular condition that has genesis in inflammation. Such a condition is caused by inflammation or has inflammation as a component to the disease state. Such ocular conditions include, but are not limited to, retinal diseases (such as diabetic retinopathy, sickle cell retinopathy, retinopathy prematurity, macular degeneration (e.g., early onset macular degeneration, neovascular macular degeneration, age-related macular degeneration)), rubeosis iritis, inflammatory diseases (e.g., uveitis, including anterior, intermediate, and posterior uveitis, chronic uveitis, ocular toxoplasmosis, toxocariasis, and pars planitis), neoplasms (retinoiplastoma, pseudoglioma), Fuchs' heterochromic iridocyclitis, neovascular glaucoma, corneal neovascularization, sequelae vascular diseases (retina ischemia, choroidal vascular insufficiency, choroidal thrombosis, carotid artery ischemia), choroidal neovascularization, ptergium, neovascularization of the optic nerve, neovascularization due to penetration of the eye or contusive ocular injury and exudative retinopathies like myopic retinopathies, cystoid macular edema arising from various etiologies, exudative macular degeneration, diabetic macular edema, central vein occlusion, and branch vein occlusion.

Non-limiting examples of said at least an anti-inflammatory medicament are the corticosteroids (e.g., glucocorticosteroids), the non-steroid anti-inflammatory drugs (“NSAIDs”), and the peroxisome proliferator-activated receptor-y (“PPARγ”) ligands, combinations thereof, and mixtures thereof.

Non-limiting examples of the glucocorticosteroids are: 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortarnate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, their physiologically acceptable salts, combinations thereof, and mixtures thereof.

Non-limiting examples of the NSAIDs are: aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), ε-acetamidocaproic acid, S-(5′-adenosyl)-L-methionine, 3-amino-4-hydroxybutyric acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, their physiologically acceptable salts, combinations thereof, and mixtures thereof.

In another aspect of the present invention, an anti-inflammatory medicament is a PPARγ-binding molecule. In one embodiment, such a PPARγ-binding molecule is a PPARγ ligand that is a PPARγ agonist. Such a PPARγ ligand binds to and activates PPARγ to modulate the expression of genes containing the appropriate peroxisome proliferator response element in its promoter region.

PPARγ agonists can inhibit the production of TNF-α and other inflammatory cytokines by human macrophages (C-Y. Jiang et al., Nature, Vol. 391, 82-86 (1998)) and T lymphocytes (A. E. Giorgini et al., Horm. Metab. Res. Vol. 31, 1-4 (1999)). More recently, the natural PPARγ agonist 15-deoxy-Δ-12,14-prostaglandin J2 (or “15-deoxy-Δ-12,14-PG J2”), has been shown to inhibit neovascularization and angiogenesis (X. Xin et al., J. Biol. Chem. Vol. 274:9116-9121 (1999)) in the rat cornea. Spiegelman et al., in U.S. Pat. No. 6,242,196, disclose methods for inhibiting proliferation of PPARγ -responsive hyperproliferative cells by using PPARγ agonists; numerous synthetic PPARγ agonists are disclosed by Spiegelman et al., as well as methods for diagnosing PPARγ-responsive hyperproliferative cells. All documents referred to herein are incorporated by reference. PPARs are differentially expressed in diseased versus normal cells. PPARγ is expressed to different degrees in the various tissues of the eye, such as some layers of the retina and the cornea, the choriocapillaris, uveal tract, conjunctival epidermis, and intraocular muscles (see, e.g., U.S. Pat. No. 6,316,465).

In one aspect, a PPARγ agonist used in a composition or a method of the present invention is a thiazolidinedione, a derivative thereof, or an analog thereof. Non-limiting examples of thiazolidinedione-based PPARγ agonists include pioglitazone, troglitazone, ciglitazone, englitazone, rosiglitazone, and chemical derivatives thereof. Other PPARγ agonists include Clofibrate (ethyl 2-(4-chlorophenoxy)-2-methylpropionate), clofibric acid (2-(4-chlorophenoxy)-2-methylpropanoic acid), GW 1929 (N-(2-benzoylphenyl)-O-{2-(methyl-2-pyridinylamino)ethyl}-L-tyrosine), GW 7647 (2-{{4-{2-({(cyclohexylamino)carbonyl}(4-cyclohexylbutyl)amino}ethyl}phenyl}thio)-2-methylpropanoic acid), and WY 14643 ({{4-chloro-6-{(2,3-dimethylphenyl)amino}-2-pyrimidinyl}thio}acetic acid). GW 1929, GW 7647, and WY 14643 are commercially available, for example, from Koma Biotechnology, Inc. (Seoul, Korea). In one embodiment, the PPARγ agonist is 15-deoxy-Δ-12, 14-PG J2.

In another aspect, an anti-inflammatory medicament suitable for a composition or a method of the present invention has low solubility in the vitreous. By “low solubility,” it is meant a solubility of less than about 50 mg/100 ml (preferably, less than about 30 mg/100 ml; more preferably, less than about 20 mg/100 ml; or even more preferably, less than about 10 mg/100 ml) at 25° C.

In one embodiment, the anti-inflammatory medicament is in a form of solid particles having a size in the range from about 10 μm to about 600 μm. Alternatively, the particle size is in the range from about 50 μm to about 400 μm (or from about 50 μm to about 200 μm). Particles having size in these ranges can be prepared by wet-milling of larger particles in a suitable inert medium with the aid of inert abrasive media (such as zirconia or alumina). Alternatively, these particles may be recrystallized from a saturated or supersaturated solution, and the particle population can be classified to obtain the desired fraction. In another embodiment, a saturated solution may be atomized and flash dried to produce micrometer-sized particles.

In one aspect of the present invention, the micrometer-sized particles are co-administered intravitreally with plasmin or an enzymatically equivalent derivative thereof. The particles are populated among the collagen fibrils at the posterior hyaloid, and can adhere to these fibrils. These adhered particles can provide inertia to the movement of the fibrils and promote their physical separation from the inner limiting membrane, thus accelerating the controlled PVD effected by the proteolytic action of plasmin or an enzymatically equivalent derivative thereof.

Methods for obtaining or producing plasmin and/or its enzymatically equivalent derivative are disclosed below.

Plasmin can be produced by activation of plasminogen precursor, which may be obtained from plasma. For example, a method of producing high-purity plasmin is disclosed in U.S. Patent Application Publication 2004/0171103 A1, which is incorporated herein by reference in its entirety. The starting material, plasminogen, can be extracted from Cohn Fraction II+III paste by affinity chromatography on Lys-SEPHAROSE™ as described by D. G. Deutsch and E. T. Mertz, “Plasminogen: purification from human plasma by affinity chromatography,” Science 170(962):1095-6 (1970). (SEPHAROSE™ is a trade name of Pharmacia, Inc., New Jersey.)

Following the extraction of plasminogen from the Cohn Fraction II+III paste, lipid and protein impurities and Transmissible Spongiform Encephalopathies (“TSE”) contaminants are reduced by precipitation with the addition polyethylene glycol (“PEG”), in a range of about 1 to about 10 percent weight/volume or the addition of about 80 to about 120 g/l ammonium sulfate. The PEG or ammonium sulfate precipitate is removed by depth filtration and the resulting solution placed on a lysine affinity resin column. The phrase “lysine affinity resin” is used generally for affinity resins containing lysine or its derivatives or ε-aminocaproic acid as the ligand. The column can be eluted with a solution having a low pH of approximately 1 to 4.

The protein obtained after elution from the affinity column is generally at least 80 percent plasminogen. The purified plasminogen is then stored at low pH in the presence of simple buffers such as glycine and lysine or ω-amino acids.

Plasminogen in solution is then activated to plasmin by the addition of a plasminogen activator, which may be accomplished in a number of ways including but not limited to streptokinase, urokinase, tissue plasminogen activator (“tPA”), or the use of urokinase immobilized on resin and use of streptokinase immobilized on resin. In one embodiment, the plasminogen activator is soluble streptokinase. The addition of stabilizers or excipients such as glycerol, ω-amino acids such as lysine, polylysine, arginine, ε-aminocaproic acid and tranexamic acid, and salt can enhance the yield of plasmin.

Plasmin can be purified from unactivated plasminogen by affinity chromatography on resin with benzamidine as the ligand and eluted preferably with a low pH solution (e.g., pH <4, or alternatively pH between about 2.5 and about 4). This step can remove essentially all degraded plasmin as well as the majority of the streptokinase.

As a polishing step for the removal of remaining streptokinase, hydrophobic interaction chromatography (“HIC”) at low pH is performed (e.g., pH <4). Following the HIC step, plasmin is formulated as a sterile protein solution by ultrafiltration and diafiltration and 0.22-μm filtration.

The eluted plasmin from such polishing step can be buffered with a low pH (e.g., pH <4), low buffering capacity agent. The low pH, low buffering capacity agent typically comprises a buffer of either an amino acid, a derivative of at least one amino acid, an oligopeptide that includes at least one amino acid, or a combination thereof. In addition, the low pH, low buffering capacity agent can comprise a buffer selected from acetic acid, citric acid, hydrochloric acid, carboxylic acid, lactic acid, malic acid, tartaric acid, benzoic acid, serine, threonine, methionine, glutamine, alanine, glycine, isoleucine, valine, alanine, aspartic acid, derivatives, and combinations thereof and mixtures thereof. The concentration of plasmin in the buffered solution can range from about 0.01 mg/ml to about 50 mg/ml of the total solution. The concentration of the buffer can range from about 1 nM to about 50 mM. Of course, these ranges may be broadened or narrowed depending upon the buffer chosen, or upon the addition of other ingredients such as additives or stabilizing agents. The amount of buffer added is typically that which will give the reversibly inactive acidified plasmin solution at a pH between about 2.5 to about 4, or between about 3 and about 3.5.

It may be advantageous to add a stabilizing or bulking agent to the reversibly inactive acidified plasmin solution obtained as disclosed above. Non-limiting examples of such stabilizing or bulking agents are polyhydric alcohols, pharmaceutically acceptable carbohydrates, salts, glucosamine, thiamine, niacinamide, and combinations thereof and mixtures thereof. The stabilizing salts can be selected from the group consisting of sodium chloride, potassium chloride, magnesium chloride, calcium chloride, and combinations thereof and mixtures thereof. Sugars or sugar alcohols may also be added, such as glucose, maltose, mannitol, sorbitol, sucrose, lactose, trehalose, and combinations thereof and mixtures thereof. Other carbohydrates that may be used are polysaccharides, such as dextrin, dextran, glycogen, starches, carboxymethylcellulose, derivatives thereof, and combinations thereof and mixtures thereof. Concentrations of a carbohydrate added to add bulk to the reversibly inactive acidified plasmin solution can be in a range from about 0.2 percent weight/volume (“% w/v”) to about 20% w/v. Concentrations for a salt, glucosamine, thiamine, niacinamide, and their combinations and mixtures can range from about 0.001 M to about 1 M.

Inactive acidified plasmin compositions including a bulking agent, such as a carbohydrate, can be optionally lyophilized at a temperature in a range, for example, from about 0° C. to about −50° C., or preferably from about 0° C. to about −20° C., to produce a powder for long-term storage.

In another aspect, plasmin or variants thereof can be produced by recombinant technology. For example, the production of recombinant microplasminogen (which can be activated to microplasmin by cleavage of the peptide bond at Arg⁵⁶¹-Val⁵⁶² using one of the plasminogen activators disclosed above) in the Pichia pastoris yeast system is disclosed in U.S. Patent Application Publication 2004/0071676 A1, which is incorporated herein by reference. Plasminogen and miniplasminogen (which also can be activated to miniplasmin by cleavage of the peptide bond at Arg⁵⁶¹-Val⁵⁶² using one of the plasminogen activators disclosed above) in the Pichia pastoris yeast system are disclosed in U.S. Patent Application Publication 2005/0124036 A1, which is incorporated herein by reference.

Recombinant plasmin or variants thereof are acidified and stored at pH less than about 5 (or alternatively less than about 4, or between about 2.5 and about 3.5). The acidified plasmin or variants thereof thus produced can further be lyophilized for long-term storage.

In one aspect, the acidified plasmin or variants thereof, produced from plasma or by recombinant technology, can be reconstituted by adding the enzyme to a formulation having a near neutral pH, to produce a formulated enzyme substantially immediately before using the enzyme.

The concentration of each of plasmin or its enzymatically equivalent derivatives in a composition of the present invention can be in the range from about 10⁻⁴ to about 5, or from about 10⁻³ to about 5, or from about 10⁻² to about 5, or from about 10⁻² to about 2, or from about 10⁻² to about 1 percent by weight.

The concentration of an anti-inflammatory medicament can be in the range from about 0.01 to about 1000 mg/ml (or, alternatively, from about 0.1 to about 500 mg/ml, or from about 1 to about 300 mg/ml, or from about 1 to about 250 mg/ml).

In one embodiment, a composition of the present invention is in a form of a suspension or dispersion. In another embodiment, the suspension or dispersion is based on an aqueous solution. For example, a composition of the present invention can comprise sterile saline solution. In still another embodiment, micrometer-sized particles of the low-solubility anti-inflammatory medicament can be coated with a physiologically acceptable surfactant (non-limiting examples are disclosed below), then the coated particles are dispersed in an aqueous medium. The coating can keep the particles in a suspension.

In a further aspect, a composition of the present invention further comprises a compound that has a function of stabilizing plasmin or its enzymatically equivalent derivatives, when present. Such a compound is hereinafter referred to as a “stabilizing agent,” which has a capability of slowing the rate of autodegradation of plasmin or its derivative in a solution; in particular, when the solution has a near neutral pH (e.g., from about 6.5 to about 8.5). The concentration of the stabilizing agent can be in the range from about 0.001 to about 5 weight percent (or alternatively, from about 0.01 to about 4, or from about 0.01 to about 2, or from about 0.01 to about 1 weight percent). The stabilizing agent can be selected from the group consisting of tranexamic acid, ε-aminocaproic acid, L-lysine, analogs of L-lysine, L-arginine, L-ornithine, γ-aminobutyric acid, glycylglycine, gelatin, human serum albumin (“HSA”), glycerin, combinations thereof, and mixtures thereof. Non-limiting examples of analogs of L-lysine include L-2-amino-3-guanidinopropionic acid, L-citruline, D-citruline, 2,6-diaminoheptanoic acid, ε,ε-dimethyl-L-lysine, α-methyl-DL-ornithine, δ-benzyloxycarbonyl-L-ornithine, (N-d-4-methyltrityl)-L-ornithine, N-δ-1-(4,4-dimethyl-2,6-dioxocyclohex-1-ylidene)ethyl)-D-ornithine, p-aminomethylbenzoic acid, and 2-aminoethylcysteine.

In another aspect, a composition of the present invention can further comprise a non-ionic surfactant, such as polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108) ), or poloxamines (synthetic block polymers of ethylene oxide and propylene oxide attached to ethylene diamine, such as those commonly known by their trade names of Tetronic®; e.g., Tetronic® 1508 or Tetronic® 908, etc., other nonionic surfactants such as Brij®, Myrj®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). Such compounds are delineated in Martindale, 34^{th} ed., pp 1411-1416 (Martindale, “The Complete Drug Reference,” S. C. Sweetman (Ed.), Pharmaceutical Press, London, 2005) and in Remington, “The Science and Practice of Pharmacy,” 21^{st} Ed., pp 291 and the contents of chapter 22, Lippincott Williams & Wilkins, New York, 2006); the contents of these sections are incorporated herein by reference. The concentration of a non-ionic surfactant, when present, in a composition of the present invention can be in the range from about 0.001 to about 5 weight percent (or alternatively, from about 0.01 to about 4, or from about 0.01 to about 2, or from about 0.01 to about 1 weight percent).

In addition, a composition of the present invention can include additives such as buffers, diluents, carriers, adjuvants, or excipients. Any pharmacologically acceptable buffer suitable for application to the eye may be used. Other agents may be employed in the composition for a variety of purposes. For example, buffering agents, preservatives, co-solvents, oils, humectants, emollients, stabilizers, or antioxidants may be employed. Water-soluble preservatives which may be employed include sodium bisulfite, sodium bisulfate, sodium thiosulfate, benzalkonium chloride, chlorobutanol, thimerosal, ethyl alcohol, methylparaben, polyvinyl alcohol, benzyl alcohol and phenylethyl alcohol. These agents may be present in individual amounts of from about 0.001 to about 5% by weight (preferably, about 0.01 % to about 2% by weight). Suitable water-soluble buffering agents that may be employed are sodium carbonate, sodium borate, sodium phosphate, sodium acetate, sodium bicarbonate, etc., as approved by the US FDA for the desired route of administration. These agents may be present in amounts sufficient to maintain a pH of the system of between about 2 to about 11. As such the buffering agent may be as much as about 5% on a weight to weight basis of the total composition. Electrolytes such as, but not limited to, sodium chloride and potassium chloride may also be included in the formulation.

In one aspect, the pH of the composition is in the range from about 6.5 to about 11. Alternatively, the pH of the composition is in the range from about 6.5 to about 9, or from about 6.5 to about 8. In another aspect, the composition comprises a buffer having a pH in one of said pH ranges.

In another aspect, the composition has a pH of about 7. Alternatively, the composition has a pH in a range from about 7 to about 7.5.

In still another aspect, the composition has a pH of about 7.4.

In yet another aspect, the composition comprises a phosphate buffer or a Tris-HCl buffer (comprising tris(hydroxymethyl)aminomethane and HCl). For example, a Tris-HCl buffer having pH of 7.4 comprises 3 g/l of tris(hydroxymethyl)aminomethane and 0.76 g/l of HCl. In yet another aspect, the buffer is 10X phosphate buffer saline (“PBS”) or 5X PBS solution.

Other buffers also may be found suitable or desirable in some circumstances, such as buffers based on HEPES (N-{2-hydroxyethyl}peperazine-N′-{2-ethanesulfonic acid}) having pKₐ of 7.5 at 25° C. and pH in the range of about 6.8-8.2; BES (N,N-bis{2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; TES (N-tris{hydroxymethyl}-methyl-2-aminoethanesulfonic acid) having pKₐ of 7.4 at 25° C. and pH in the range of about 6.8-8.2; MOBS (4-{N-morpholino}butanesulfonic acid) having pKₐ of 7.6 at 25° C. and pH in the range of about 6.9-8.3; DIPSO (3-(N,N-bis{2-hydroxyethyl}amino)-2-hydroxypropane) ) having pKₐ of 7.52 at 25° C. and pH in the range of about 7-8.2; TAPSO (2-hydroxy-3{tris(hydroxymethyl)methylamino}-1-propanesulfonic acid) ) having pKₐ of 7.61 at 25° C. and pH in the range of about 7-8.2; TAPS ({(2-hydroxy-1,1-bis(hydroxymethyl)ethyl)amino}-1-propanesulfonic acid)) having pKₐ of 8.4 at 25° C. and pH in the range of about 7.7-9.1; TABS (N-tris(hydroxymethyl)methyl-4-aminobutanesulfonic acid) having pKₐ of 8.9 at 25° C. and pH in the range of about 8.2-9.6; AMPSO (N-(1,1-dimethyl-2-hydroxyethyl)-3-amino-2-hydroxypropanesulfonic acid) ) having pKₐ of 9.0 at 25° C. and pH in the range of about 8.3-9.7; CHES (2-cyclohexyamino)ethanesulfonic acid) having pKₐ of 9.5 at 25° C. and pH in the range of about 8.6-10.0; CAPSO (3-(cyclohexylamino)-2-hydroxy-1-propanesulfonic acid) having pKₐ of 9.6 at 25° C. and pH in the range of about 8.9-10.3; or CAPS (3-(cyclohexylamino)-1-propane sulfonic acid) having pKₐ of 10.4 at 25° C. and pH in the range of about 9.7-11.1.

In another aspect, the present invention provides a method for producing a composition for use in inducing a controlled PVD, the method comprising adding plasmin or an enzymatically equivalent thereof to at least an anti-inflammatory medicament. In one embodiment, the method further comprises mixing together the ingredients of the composition. In another embodiment, said at least anti-inflammatory medicament is a corticosteroid, an NSAID, or a PPARγ agonist. In still another embodiment, the mixing is carried out in a medium comprising a buffer having a pH in the range from about 6.5 to about 8.5. In still another embodiment, said at least anti-inflammatory medicament is in the form of solid particles having a size in the range from about 10 μm to about 600 μm. In yet another embodiment, said at least anti-inflammatory medicament is triamcinolone acetonide.

In another aspect, the present invention provides a method for producing a composition for use in inducing a controlled PVD, the method comprising: (a) storing plasmin or an enzymatically equivalent derivative thereof at a pH less than about 5; and (b) adding said stored plasmin or enzymatically equivalent derivative thereof to a formulation that comprises at least an anti-inflammatory medicament.

In a further aspect of the method, said at least an anti-inflammatory medicament is selected from the group consisting of the glucocorticosteroids, the NSAIDs, and the PPARγ ligands, combinations thereof, and mixtures thereof. In one embodiment, said anti-inflammatory medicament has low solubility in the vitreous and is in the form of micrometer-sized particles in the range from about 10 μm to about 600 μm (or, alternatively, from about 10 μm to about 400 μm, or from about 50 μm to about 400 μm, or from about 50 μm to about 200 μm).

In one embodiment, the formulation further comprises a buffer having a pH in the range from about 6.5 to about 11 (or alternatively, from about 6.5 to about 9, or from about 6.5 to about 8).

In another aspect, the formulation further comprises a stabilizing agent selected from the group consisting of tranexamic acid, ε-aminocaproic acid, L-lysine, analogs of L-lysine, L-arginine, L-ornithine, γ-aminobutyric acid, glycylglycine, gelatin, HSA, glycerin, combinations thereof, and mixtures thereof. Non-limiting examples of analogs of L-lysine include L-2-amino-3-guanidinopropionic acid, L-citruline, D-citruline, 2,6-diaminoheptanoic acid, ε,ε-dimethyl-L-lysine, α-methyl-DL-ornithine, δ-benzyloxycarbonyl-L-ornithine, (N-d-4-methyltrityl)-L-ornithine, N-δ-1-(4,4-dimethyl-2,6-dioxocyclohex-1-ylidene)ethyl)-D-ornithine, p-aminomethylbenzoic acid, and 2-aminoethylcysteine.

In another embodiment, the formulation further comprises a non-ionic surfactant. Non-limiting examples of suitable non-ionic surfactants are disclosed above.

In another aspect, the step of storing of said plasmin or enzymatically equivalent derivative thereof is carried out at a pH less than about 4.5. Alternatively, said pH is less than 4 or in the range from about 2.5 to about 4.5, or from about 2.5 to about 4, or from about 3 to about 4.

In still another aspect, the present invention is useful in producing a composition comprising active plasmin or an enzymatically equivalent derivative thereof after prolonged storage after its manufacture for use in inducing a controlled PVD in a patient in need thereof.

In yet another aspect, a composition of the present invention can induce a controlled PVD to prevent, stop, reduce, or ameliorate at least an effect or complication of an ocular condition that initiates the need of such a controlled PVD.

In a further aspect, a composition of the present invention also can effect a treatment, stoppage, reduction, or amelioration of at least a potential cause of an ocular condition that initiates the need of such a controlled PVD.

In still another aspect, the present invention provides a kit for making a composition for use in inducing a controlled PVD. The composition comprises plasmin or an enzymatically equivalent derivative thereof and at least an anti-inflammatory medicament. The kit comprises: (a) plasmin or said enzymatically equivalent derivative thereof that has been preserved at a pH less than about 5; and (b) a formulation that comprises said at least an anti-inflammatory medicament, said formulation being provided in a separate container or package. In one embodiment, said at least an anti-inflammatory medicament is selected from the groups of anti-inflammatory medicaments disclosed above. In another embodiment, the formulation comprises said at least an anti-inflammatory medicament in the form of solid particles having a size in the range from about 10 μm to about 600 μm, the solid particles being dispersed in a liquid medium.

In still another embodiment, the formulation further comprises a stabilizing agent selected from the group consisting of tranexamic acid, ε-aminocaproic acid, L-lysine, analogs of L-lysine, L-arginine, L-ornithine, γ-aminobutyric acid, glycylglycine, gelatin, HSA, glycerin, combinations thereof, and mixtures thereof. Non-limiting examples of analogs of L-lysine include L-2-amino-3-guanidinopropionic acid, L-citruline, D-citruline, 2,6-diaminoheptanoic acid, ε,ε-dimethyl-L-lysine, α-methyl-DL-ornithine, δ-benzyloxycarbonyl-L-ornithine, (N-d-4-methyltrityl)-L-ornithine, N-δ-1-(4,4-dimethyl-2,6-dioxocyclohex-1-ylidene)ethyl)-D-ornithine, p-aminomethylbenzoic acid, and 2-aminoethylcysteine.

In still another aspect, the present invention provides a method for inducing a controlled PVD in an eye of a patient, the method comprising: (a) providing a composition that comprises plasmin or an enzymatically equivalent derivative thereof and at least an anti-inflammatory medicament; and (b) administering said composition into the vitreous humor of the eye, thereby inducing said controlled PVD in said eye. In one embodiment, said at least an anti-inflammatory medicament is selected from the group consisting of the glucocorticosteroids, the NSAIDs, and the PPARγ ligands, combinations thereof, and mixtures thereof. In one embodiment, said anti-inflammatory medicament has low solubility in the vitreous and is in the form of micrometer-sized particles in the range from about 10 μm to about 600 μm. In another embodiment, the composition is in the form of a suspension or dispersion.

In one embodiment, the composition further comprises a buffer having a pH in the range from about 3 to about 11 (or alternatively, from about 3 to about 9, or from about 3 to about 8).

In another embodiment, the composition further comprises a compound selected from the group consisting of tranexamic acid, ε-aminocaproic acid, L-lysine, analogs of L-lysine, L-arginine, L-ornithine, γ-aminobutyric acid, glycylglycine, gelatin, HSA, glycerin, combinations thereof, and mixtures thereof. Non-limiting examples of analogs of L-lysine include L-2-amino-3-guanidinopropionic acid, L-citruline, D-citruline, 2,6-diaminoheptanoic acid, ε,ε-dimethyl-L-lysine, α-methyl-DL-ornithine, δ-benzyloxycarbonyl-L-ornithine, (N-d-4-methyltrityl)-L-ornithine, N-δ-1-(4,4-dimethyl-2,6-dioxocyclohex-1-ylidene)ethyl)-D-ornithine, p-aminomethylbenzoic acid, and 2-aminoethylcysteine.

Non-limiting amounts or concentrations of the various materials or compounds disclosed above are also applicable to the various methods of the present invention disclosed herein.

In a further aspect, the step of providing said composition comprises: (i) providing said plasmin or said enzymatically equivalent derivative thereof that has been preserved at a pH less than about 5; (ii) providing said at least an anti-inflammatory medicament; and (iii) producing said composition from said plasmin or said enzymatically equivalent derivative thereof and said at least an anti-inflammatory medicament.

In still another aspect, the patient may be one who has one or more symptoms of the beginning of a pathological PVD and the method induces a controlled PVD. Such a controlled PVD can arrest or prevent damage to the retina, which would occur if the pathological uncontrolled PVD is allowed to continue.

In another embodiment, said composition is administered in an amount containing a therapeutically effective amount of plasmin or an enzymatically equivalent derivative thereof to induce said controlled PVD.

Method of injecting plasmin or derivatives thereof into eye for controlled PVD is now described.

A composition comprising plasmin or an enzymatically equivalent derivative thereof and at least an anti-inflammatory medicament can be injected intravitreally, for example through the pars plana of the ciliary body, to induce controlled PVD using a fine-gauge needle, such as 25-30 gauge. Administration of such a composition can be used to prevent, treat, or ameliorate the potentially blinding complications of an ocular condition, such as diabetic retinopathy, retinal detachment, macular edema, macular hole, and retinal tears. Typically, an amount from about 25 μl to about 200 μl of a composition comprising about 1-5 IU of plasmin or derivatives thereof per 50 μl of formulation is administered into the vitreous. Alternatively, a composition can comprise about 0.001-50 mg/ml (or about 0.2-20 mg/ml, or about 0.2-10 mg/ml, or about 0.5-8 mg/ml) of plasmin or derivatives thereof. Such administration of plasmin or derivatives thereof may be repeated to achieve a full effect upon assessment of the treatment results and recommendation by a skilled medical practitioner.

Tables 1-16 show non-limiting examples of compositions of the present invention, which can be used in the practice of the methods of the present invention disclosed above.

TABLE 1

| | | |
|---|---|---|
| Ingredient | Amount per ml | % composition |

| | | | |
|---|---|---|---|
| Plasmin | 2 | mg | 0.2 |
| Trehalose | 20 | mg | 2 |
| sodium acetate | 2.4 | mg | 0.24 |
| ε-amino caproic acid | 3.0 | mg | 0.3 |
| triamcinolone | 2 | mg | 0.2 |
| acetonide | | | |
| normal saline | QS to 1 | ml | 97.06 |

TABLE 2

| | | |
|---|---|---|
| Ingredient | Amount per ml | % composition |

| | | | |
|---|---|---|---|
| plasmin | 2 | mg | 0.2 |
| trehalose | 20 | mg | 2 |
| sodium acetate | 2.4 | mg | 0.24 |
| ε-amino caproic acid | 3.0 | mg | 0.3 |
| prednisolone | 2 | mg | 0.2 |
| normal saline | QS to 1 | ml | 97.06 |

TABLE 3

| | | |
|---|---|---|
| Ingredient | Amount per ml | % composition |

| | | | |
|---|---|---|---|
| plasmin | 4 | mg | 0.4 |
| trehalose | 20 | mg | 2 |
| sodium acetate | 2.4 | mg | 0.24 |
| ε-amino caproic acid | 3.0 | mg | 0.3 |
| loteprednol | 2 | mg | 0.2 |
| etabonate | | | |
| normal saline | QS to 1 | ml | 96.86 |

TABLE 4

| | | |
|---|---|---|
| Ingredient | Amount per ml | % composition |

| | | | |
|---|---|---|---|
| plasmin | 2 | mg | 0.2 |
| trehalose | 20 | mg | 2 |
| sodium acetate | 2.4 | mg | 0.24 |
| tranexamic acid | 3.0 | mg | 0.3 |
| difluprednate | 2 | mg | 0.2 |
| normal saline | QS to 1 | ml | 97.06 |

TABLE 5

| | | |
|---|---|---|
| Ingredient | Amount per ml | % composition |

| | | | |
|---|---|---|---|
| plasmin | 10 | mg | 1 |
| trehalose | 20 | mg | 2 |
| sodium acetate | 2.4 | mg | 0.24 |
| ε-amino caproic acid | 3.0 | mg | 0.3 |
| ibuprofen | 5 | mg | 0.5 |
| normal saline | QS to 1 | ml | 95.96 |

TABLE 6

| | | |
|---|---|---|
| Ingredient | Amount per ml | % composition |

| | | | |
|---|---|---|---|
| microplasmin | 2 | mg | 0.2 |
| trehalose | 20 | mg | 2 |
| sodium acetate | 2.4 | mg | 0.24 |
| ε-amino caproic acid | 3.0 | mg | 0.3 |
| triamcinolone | 3 | mg | 0.3 |
| acetonide | | | |
| normal saline | QS to 1 | ml | 96.96 |

TABLE 7

| | | |
|---|---|---|
| Ingredient | Amount per ml | % composition |

| | | | |
|---|---|---|---|
| miniplasmin | 2 | mg | 0.2 |
| trehalose | 20 | mg | 2 |
| sodium citrate | 2.4 | mg | 0.24 |
| tranexamic acid | 3.0 | mg | 0.3 |
| loteprednol | 5 | mg | 0.5 |
| etabonate | | | |
| normal saline | QS to 1 | ml | 96.76 |

TABLE 8

| | | |
|---|---|---|
| Ingredient | Amount per ml | % composition |

| | | | |
|---|---|---|---|
| a truncated plasmin | 2 | mg | 0.2 |
| consisting essentially | | | |
| of kringle-1 domain | | | |
| and enzymatic | | | |
| domain of plasmin | | | |
| mannitol | 20 | mg | 2 |
| sodium acetate | 2.4 | mg | 0.24 |
| ε-amino caproic acid | 3.0 | mg | 0.3 |
| triamcinolone | 5 | mg | 0.5 |
| acetonide | | | |
| normal saline | QS to 1 | ml | 96.76 |

TABLE 9

| | | |
|---|---|---|
| Ingredient | Amount per ml | % composition |

| | | | |
|---|---|---|---|
| plasmin | 2 | mg | 0.2 |
| trehalose | 20 | mg | 2 |
| sodium acetate | 2.4 | mg | 0.24 |
| e-amino caproic acid | 3.0 | mg | 0.3 |
| 15-deoxy-Δ-12,14- | 2 | mg | 0.2 |
| PG J2 | | | |
| normal saline | QS to 1 | ml | 97.06 |

TABLE 10

| | | |
|---|---|---|
| Ingredient | Amount per ml | % composition |

| | | | |
|---|---|---|---|
| plasmin | 2 | mg | 0.2 |
| trehalose | 20 | mg | 2 |
| sodium acetate | 2.4 | mg | 0.24 |
| ε-amino caproic acid | 3.0 | mg | 0.3 |
| ciglitazone | 3 | mg | 0.3 |
| normal saline | QS to 1 | ml | 96.96 |

TABLE 11

| | | |
|---|---|---|
| Ingredient | Amount per ml | % composition |

| | | | |
|---|---|---|---|
| plasmin | 2 | mg | 0.2 |
| mannitol | 20 | mg | 2 |
| sodium acetate | 2.4 | mg | 0.24 |
| ε-amino caproic acid | 3.0 | mg | 0.3 |
| clofibrate | 6 | mg | 0.6 |
| normal saline | QS to 1 | ml | 96.66 |

TABLE 12

| | | |
|---|---|---|
| Ingredient | Amount per ml | % composition |

| | | | |
|---|---|---|---|
| plasmin | 2 | mg | 0.2 |
| trehalose | 20 | mg | 2 |
| sodium acetate | 2.4 | mg | 0.24 |
| ε-amino caproic acid | 3.0 | mg | 0.3 |
| rosiglitazone | 2 | mg | 0.2 |
| loteprednol etabonate | 5 | mg | 0.5 |
| normal saline | QS to 1 | ml | 96.56 |

TABLE 13

| | | |
|---|---|---|
| Ingredient | Amount per ml | % composition |

| | | | |
|---|---|---|---|
| miniplasmin | 2 | mg | 0.2 |
| trehalose | 20 | mg | 2 |
| sodium acetate | 2.4 | mg | 0.24 |
| ε-amino caproic acid | 3.0 | mg | 0.3 |
| triamcinolone acetonide | 5 | mg | 0.5 |
| 15-deoxy-Δ-12,14-PG J2 | 3 | mg | 0.3 |
| normal saline | QS to 1 | ml | 96.46 |

TABLE 14

| | | |
|---|---|---|
| Ingredient | Amount per ml | % composition |

| | | | |
|---|---|---|---|
| microplasmin | 5 | mg | 0.5 |
| trehalose | 20 | mg | 2 |
| sodium citrate | 2.4 | mg | 0.24 |
| tranexamic acid | 3.0 | mg | 0.3 |
| triamcinolone acetonide | 5 | mg | 0.5 |
| ciglitazone | 3 | mg | 0.3 |
| phosphate buffer (pH 7.4) | QS to 1 | ml | ˜96.1 |

TABLE 15

| | | |
|---|---|---|
| Ingredient | Amount per ml | % composition |

| | | | |
|---|---|---|---|
| miniplasmin | 2 | mg | 0.2 |
| mannitol | 20 | mg | 2 |
| sodium acetate | 2.4 | mg | 0.24 |
| ε-amino caproic acid | 3 | mg | 0.3 |
| predsinolone | 5 | mg | 0.5 |
| troglitazone | 3 | mg | 0.3 |
| normal saline | QS to 1 | ml | 96.46 |

TABLE 16

| | | |
|---|---|---|
| Ingredient | Amount per ml | % composition |

| | | | |
|---|---|---|---|
| plasmin | 2 | mg | 0.2 |
| mannitol | 20 | mg | 2 |
| sodium acetate | 2.4 | mg | 0.24 |
| L-lysine | 2 | mg | 0.2 |
| predsinolone | 5 | mg | 0.5 |
| troglitazone | 3 | mg | 0.3 |
| aspirin | 1 | mg | 0.1 |
| normal saline | QS to 1 | ml | 96.46 |

Experimental Study of Efficacy of a Composition of the Present Invention for PVD

The purpose of this ex vivo study was to compare the efficacy of 200 μg human-derived plasmin (Bausch & Lomb Incorporated's compound name “BOL-303209-X”) alone and in combination with triamcinolone acetonide (“TA,” a glucocorticoid anti-inflammatory medicament) on PVD in Dutch Belted Rabbits using scanning electron microscopy. This study was designed to use intravitreal injection of TA to enhance clinical observations for clear identification of PVD, to minimize possible inflammatory responses, and to assess whether improved efficacy could be achieved by the combined use of BOL-303209-X and TA.

Experimental Design

Twelve (12) normal eyes from six (6) Dutch Belted Rabbits (both male and female) were used. All right eyes from 6 rabbits were injected with BOL-303209-X plus vehicle (Group 1, n=6 eyes) and all left eyes were injected with BOL-303209-X plus TA (Group 2, n=3, 2 mg TA/eye; Group 3, n=3, 4 mg TA/eye). BOL-303209-X was freshly prepared and kept on ice before use.

On the day of operation, rabbits were anesthetized, eyes disinfected and pupils dilated 15-20 minutes prior to injection. Each rabbit received two intravitreal injections. In Groups 1, 2, and 3, an initial 50-μl injection of BOL-303209-X at a dose of 200 μg per eye in a formulation of 5 mM epsilon-aminocaproic acid (“EACA”)/0.05% Tween 80/0.9% saline was given intravitreally in both the right and left eyes. In Group 1 (Control) a second 50-μl injection of formulation vehicle (0.05% Tween 80/0.05% hydroxypropyl methylcellulose (HPMC)/0.9% saline) was delivered in the right eyes 5 minutes after the first injection. In Groups 2 and 3, a formulation containing TA at a dose of either 2 mg (Group 2) or 4 mg (Group 3) was injected into the left eyes in the same way. Injections were performed approximately 3-4 mm posterior to the limbus in the inferior temporal quadrant of the right eye and the inferior nasal quadrant of the left eye with a 30-gauge needle under an operating microscope. Test articles were delivered into the mid-inferior vitreous.

Eyes were examined with a slit-lamp biomicroscope assisted with a Volk Lens at 4 days prior to injection for baseline documentation. To evaluate the efficacy of BOL-303209-X, all eyes were directly observed under a surgical microscope for 5 minutes after injection for possible changes in the vitreo-retina. The injected eyes were followed up at 7 and 14 days after injection. Attention was paid particularly to the vitreoretinal interface for signs of PVD and to the retina for signs of toxicity during slit-lamp examination.

Fourteen days after injection the in-life phase of the study was terminated, the rabbits were euthanized by over dose of pentobarbital, their eyes enucleated and fixed in Karnovsky's solution for at least 24 hours. The fixed eyes were processed further for SEM. The posterior pole of each eye was examined. Micrographs at magnifications ranging from 100-2,500× of each specimen or up to 10,000× in some cases were examined to assess the extent of posterior vitreous detachment. The amount of residual vitreous was scored on a scale of 0-4 with a score of 4 representing complete removal of vitreous from the inner retina according to a standard protocol.

TABLE I
Design summary

| | First Injection | | | Second Injection | | |
|---|---|---|---|---|---|---|
| | Test Article/ | Dose | Dose | Test Article/ | Dose | Dose |
| Group | Placebo | Concentration | per eye | Placebo | Concentration | per eye |
| 1-OD | 200 μg BOL- | 4 mg/mL | 200 μg/50 μL | 0.05% Tween | — | — |
| n = 6 | 303209-X in 5 | | | 80/0.5% HPMC/ | | |
| | mM EACA + | | | 0.9% Saline | | |
| 2-OS | 0.05% Tween | | | TA in 0.05% | 40 mg/mL | 2 mg/50 μL |
| n = 3 | 80 in 0.9% | | | Tween 80/0.5% | | |
| | Saline | | | HPMC/0.9% | | |
| | | | | Saline | | |
| 3-OS | | | | | 80 mg/mL | 4 mg/50 μL |
| n = 3 | | | | | | |

| Group | Study Animal number (eye) |
|---|---|
| 1 | 1 (OD), 2 (OD), 3 (OD), 4 (OD), 5 (OD), 6 (OD) |
| 2 | 1 (OS), 2 (OS), 3 (OS) |
| 3 | 4 (OS), 5 (OS), 6 (OS) |

Schedule

| | |
|---|---|
| Baseline | Feb. 17, 2006 |
| Injection | Feb. 22, 2006 |
| Observations | Day 7 (Mar. 1, 2006), Day 14 (Mar. 8, 2006) |
| Sacrifice, enucleation, fixation | Day 14 (Mar. 8, 2006) |

Sample Preparation

After enucleation, the eyes were trimmed of external muscle, fat, and connective tissue, and cut using a razor blade about 3 mm behind the limbus to facilitate penetration of fixative. The eyes were placed in ice-cold fixative consisting of 2% formaldehyde (from paraformaldehyde), 2.5% glutaraldehyde, in 0.1 phosphate buffer, with 0.1 M sucrose and 0.5 mM CaCl₂ added, pH 7.2. Each eye was immersed in approximately 40 ml fixative. After one hour, the fixative was exchanged with fresh fixative and the eyes were transported to B&L and stored refrigerated in fixative.

SEM Processing

Tissue specimen that included sclera, choroid, retina with the optic nerve (approximately 1 cm in diameter) were surgically removed from each eye. These were dehydrated though a graded ethanol series (30%, 50%, 70%, 85%, 95%, 100%) for thirty minutes per dilution. Dehydrated samples were dried to the critical point (Samdri-PVT-3B, Tousimis, Rockville, Md.) and mounted on aluminum SEM pucks with colloidal graphite (Ted Pella, Inc, Redding, Calif.). Mounted samples were sputter coated with 15 nm gold/palladium (Hummer X, Anatech LTD, Alexandria, Va.). The specimens were imaged with a scanning electron microscope (Model Quanta 400, FEI, Hillsboro, Oreg.).

SEM Evaluation

Micrographs of each specimen were reviewed and assessed for the presence of residual vitreous. Twelve fields were photographed—three above the optic nerve, three including the optic nerve and the adjacent nasal and temporal medullary rays, three inferior to the optic nerve and three below those. Each of the inferior fields was graded by one investigator according to the following grading system (Table II) and the average calculated. The number of eyes in each group with a score greater than 3 was determined. The group with the highest percentage of eyes with a grade 3 score or higher was considered the best formulation.

TABLE II
Grading System for SEM Specimens

Grade 0 - extensive amount of residual vitreous
Grade 1 - moderate amount of residual vitreous
Grade 2 - mild amount of residual vitreous
Grade 3 - Inner retina visible through sparse collagen fibrils
Grade 4 - None to trace collagen fibrils covering inner retina

Data Analysis

The mean score and standard deviation of each group was determined. The group with the highest mean score was considered to be the most effective formulation.

Results

The mean score for the control eyes (n=6) treated with an initial injection of 50-μl of BOL-303209-X at a dose of 200 μg per eye and a second injection of 50-μl of formulation vehicle (0.05% Tween 80/0.05% HPMC/0.9% saline) was 2.6 (Table III). The mean score for the eyes (n=3) treated with an initial injection of 50-μl of BOL-303209-X at a dose of 200 μg per eye and a second injection of 50-μl of TA at a dose of 2 mg per eye was 3.3 (Table IV). The mean score for the eyes (n=3) treated with an initial injection of 50-μl of BOL-303209-X at a dose of 200 μg per eye and a second injection of 50-μl of TA at a dose of 4 mg per eye was 3.3 (Table 4). The mean score for the eyes (n=6) treated with an initial injection of 50-μl of BOL-303209-X at a dose of 200 μg per eye and a second injection of 50-μl of TA at either a dose of 2 or 4 mg per eye was also 3.3 (Table 4). The results indicate that the 2 mg and 4 mg TA doses had equivalent effects.

TABLE III
Score of vitreo-retinal specimens at 6
inferior fields of eyes from Group 1

| OP | IN | INL | IC | ICL | IT | ITL | Mean | n ≧ 3 |
|---|---|---|---|---|---|---|---|---|
| 24 | 3 | 3 | 3 | 1 | 3 | 2 | 2.5 | |
| 25 | 2 | 1 | 3 | 1 | 2 | 1 | 1.7 | |
| 26 | 3 | 3 | 3 | 1 | 3 | 2 | 2.5 | |
| 27 | 3 | 3 | 3 | 3 | 3 | 3 | 3.0 | 1 |
| 28 | 3 | 3 | 4 | 3 | 3 | 3 | 3.2 | 1 |
| 29 | 3 | 3 | 3 | 2 | 3 | 3 | 2.8 | |
| | | | | | | Mean | 2.6 | 2 |
| | | | | | | SD | 0.5 | |

IN—Infra-Nasal Area
INL—Infra-Nasal Inferior Area
IC—Infra-Central Area
ICL—Infra-Central Lower Area
IT—Infra-Temporal Area
ITL—Infra-Temporal Lower Area

TABLE IV
Score of vitreo-retinal specimens at 6 inferior
fields of eyes from Groups 2 and 3.

| | TA | | | | | | | | n ≧ |
|---|---|---|---|---|---|---|---|---|---|
| OS | (mg) | IN | INL | IC | ICL | IT | ITL | Mean | 3 |
| Group 2 | | | | | | | | | |
| 24 | 2 | 4 | 3 | 3 | 3 | 3 | 3 | 3.2 | 1 |
| 25 | 2 | 3 | 3 | 3 | 3 | 3 | 3 | 3.0 | 1 |
| 26 | 2 | 3 | 4 | 4 | 4 | 3 | 4 | 3.7 | 1 |
| | | | | | | | Mean of 3 | 3.3 | |
| | | | | | | | SD | 0.4 | |
| Group 3 | | | | | | | | | |
| 27 | 4 | 3 | 3 | 3 | 3 | 3 | 3 | 3.0 | 1 |
| 28 | 4 | 3 | 3 | 4 | 3 | 4 | 3 | 3.3 | 1 |
| 29 | 4 | 4 | 3 | 4 | 3 | 4 | 3 | 3.5 | 1 |
| | | | | | | | Mean of 3 | 3.3 | |
| | | | | | | | SD | 0.3 | |
| | | | | | | All | Mean of 6 | 3.3 | 6 |
| | | | | | | eyes | SD | 0.3 | |

### CONCLUSION

The results based on examination of the posterior pole using scanning electron microscopy indicate that the extent of posterior vitreous detachment was greater in those eyes treated with a combination of human plasma-derived plasmin (BOL-303209-X) and either 2 mg or 4 mg triamcinolone acetonide compared to those eyes treated with BOL-303209-X only.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A composition comprising: (a) plasmin or an enzymatically equivalent derivative thereof; and (b) at least an anti-inflammatory medicament.

2. The composition of claim 1, wherein said at least an anti-inflammatory medicament is selected from the group consisting of corticosteroids, non-steroid anti-inflammatory drugs (“NSAIDs”), peroxisome proliferator-activated receptor-γ (“PPARγ”) ligands, combinations thereof, and mixtures thereof.

3. The composition of claim 1, wherein said at least an anti-inflammatory medicament is in a form of solid particles having a size in a range from about 10 μm to about 600 μm.

4. The composition of claim 1, wherein said at least an anti-inflammatory medicament is in a form of solid particles having a size in a range from about 50 μm to about 400 μm.

5. The composition of claim 1, wherein said at least an anti-inflammatory medicament is selected from the group consisting of 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortarnate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, physiologically acceptable salts thereof, combinations thereof, and mixtures thereof.

6. The composition of claim 1, wherein said at least an anti-inflammatory medicament is selected from the group consisting of aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, ε-acetamidocaproic acid, S-(5′-adenosyl)-L-methionine, 3-amino-4-hydroxybutyric acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, their physiologically acceptable salts, combinations thereof, and mixtures thereof.

7. The composition of claim 1, wherein said at least an anti-inflammatory medicament is selected from the group consisting of enfenamic acid, etofenamate, flufenamic acid, isonixin, meclofenamic acid, mefenamic acid, niflumic acid, talniflumate, terofenamate, tolfenamic acid, aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac, bumadizon, butibufen, fenbufen, xenbucin, clidanac, ketorolac, tinoridine, alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen, difenamizole, epirizole, apazone, benzpiperylon, feprazone, mofebutazone, morazone, oxyphenbutazone, phenylbutazone, pipebuzone, propyphenazone, ramifenazone, suxibuzone, thiazolinobutazone, acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine, ampiroxicam, droxicam, isoxicam, lornoxicam, piroxicam, tenoxicam, ε-acetamidocaproic acid, S-(5′-adenosyl)-L-methionine, 3-amino4-hydroxybutyric acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, their physiologically acceptable salts, combinations thereof, and mixtures thereof.

8. The composition of claim 1, wherein said at least an anti-inflammatory medicament is selected from the group consisting of thiazolidinedione; derivatives thereof; analogs thereof; ethyl 2-(4-chlorophenoxy)-2-methylpropionate); clofibric acid; N-(2-benzoylphenyl)-O-{2-(methyl-2-pyridinylamino)ethyl}-L-tyrosine; 2-{{4-{2-{{(cyclohexylamino)carbonyl}(4-cyclohexylbutyl)amino}ethyl}phenyl}thio}-2-methylpropanoic acid; {{4-chloro-6-{(2,3-dimethylphenyl)amino}-2-pyrimidinyl}thio}acetic acid; 15-deoxy-Δ-12,14-PG J2; combinations thereof; and mixtures thereof.

9. The composition of claim 1, wherein the enzymatically equivalent derivative of plasmin is selected from the group consisting of microplasmin, miniplasmin, truncated forms of plasmin, variants of plasmin, combinations thereof, and mixtures thereof.

10. The composition of claim 1, wherein the composition further comprises a stabilizing agent for said plasmin or said enzymatically equivalent derivative thereof.

11. The composition of claim 10, wherein the stabilizing agent is selected from the group consisting of tranexamic acid, ε-aminocaproic acid, L-lysine, analogs of L-lysine, L-arginine, L-ornithine, γ-aminobutyric acid, glycylglycine, gelatin, human serum albumin (“HSA”), glycerin, combinations thereof, and mixtures thereof.

12. The composition of claim 11, wherein the L-lysine analogs are selected from the group consisting of L-2-amino-3-guanidinopropionic acid, L-citruline, D-citruline, 2,6-diaminoheptanoic acid, ε,ε-dimethyl-L-lysine, α-methyl-DL-ornithine, δ-benzyloxycarbonyl-L-ornithine, (N-d-4-methyltrityl)-L-ornithine, N-δ-1-(4,4-dimethyl-2,6-dioxocyclohex-1-ylidene)ethyl)-D-ornithine, p-aminomethylbenzoic acid, and 2-aminoethylcysteine.

13. A composition comprising: (a) plasmin or an enzymatically equivalent derivative thereof; and (b) at least an anti-inflammatory medicament selected from the group consisting of corticosteroids, NSAIDs, PPARγ agonists, combinations thereof, and mixtures thereof;
wherein the enzymatically equivalent derivative of plasmin is selected from the group consisting of microplasmin, miniplasmin, truncated plasmin, combinations thereof, and mixtures thereof; said at least an anti-inflammatory medicament is in a form of solid particles having a size in range from about 10 μm to about 600 μm; and the composition is in a form of a suspension.

14. The composition of claim 13, wherein at least an anti-inflammatory medicament has a solubility in a vitreous of less than about 50 mg/100 ml.

15. The composition of claim 13, wherein a concentration of each of said plasmin, said enzymatically equivalent derivative of plasmin, and said at least an anti-inflammatory medicament is in a range from about 10⁻⁴ to about 5 weight percent.

16. The composition of claim 13, further comprising a stabilizing agent for said plasmin or for said enzymatically equivalent derivative thereof.

17. A method for producing a composition for use in inducing a controlled posterior vitreous detachment (“PVD”), the method comprising:
(a) providing plasmin or an enzymatically equivalent derivative thereof; and
(b) adding said plasmin or enzymatically equivalent derivative thereof to at least an anti-inflammatory medicament.

18. The method of claim 17, wherein said plasmin or an enzymatically equivalent derivative thereof has been preserved at a pH less than about 5.

19. The method of claim 17, further comprising adding a stabilizing agent selected from the group consisting of tranexamic acid, ε-aminocaproic acid, L-lysine, analogs of L-lysine, L-arginine, L-ornithine, γ-aminobutyric acid, glycylglycine, gelatin, HSA, glycerin, combinations thereof, and mixtures thereof.

20. The method of claim 17, wherein said at least an anti-inflammatory medicament is selected from the group consisting of corticosteroids, NSAIDs, PPARγ agonists, combinations thereof, and mixtures thereof.

21. Use of plasmin or an enzymatically equivalent derivative thereof and at least an anti-inflammatory medicament, to produce a composition for inducing a controlled PVD in a subject in need therefor.

22. The use of claim 21, wherein said at least an anti-inflammatory medicament is selected from the group consisting of corticosteroids, NSAIDs, PPARγ agonists, combinations thereof, and mixtures thereof.

23. A method for inducing a controlled PVD in an eye of a patient, the method comprising:
(a) providing a composition that comprises plasmin or an enzymatically equivalent derivative thereof and at least an anti-inflammatory medicament, which is selected from the group consisting of corticosteroids, NSAIDs, PPARγ agonists, combinations thereof, and mixtures thereof; and
(b) administering said composition to or into the vitreous humor of the eye, thereby inducing said controlled PVD in said eye.

24. The method of claim 23, wherein said composition is in a form of a suspension of micrometer-sized particles of said an anti-inflammatory medicament in a liquid medium comprising said plasmin or said enzymatically equivalent derivative thereof.

25. The method of claim 23, wherein said enzymatically equivalent derivative of plasmin is selected from the group consisting of microplasmin, miniplasmin, truncated forms of plasmin, combinations thereof, and mixtures thereof.

26. The method of claim 23, wherein the composition further comprises a stabilizing agent for said plasmin or said enzymatically equivalent derivative thereof.

27. The method of claim 23, wherein said plasmin or enzymatically equivalent derivative thereof has been preserved at a pH less than about 5.

28. The method of claim 23, wherein said controlled PVD is induced to prevent, treat, or ameliorate at least a potential complication of an ocular condition selected from the group consisting of diabetic retinopathy, sickle cell retinopathy, retinopathy prematurity, early onset macular degeneration, neovascular macular degeneration, age-related macular degeneration, rubeosis iritis, anterior uveitis, intermediate uveitis, posterior uveitis, chronic uveitis, ocular toxoplasmosis, toxocariasis, pars planitis, retinoiplastoma, pseudoglioma, Fuchs' heterochromic iridocyclitis, neovascular glaucoma, corneal neovascularization, retina ischemia, choroidal vascular insufficiency, choroidal thrombosis, carotid artery ischemia, choroidal neovascularization, ptergium, neovascularization of optic nerve, neovascularization due to penetration of the eye, neovascularization due to contusive ocular injury, exudative retinopathies, exudative macular degeneration, diabetic macular edema, central vein occlusion, branch vein occlusion, and combinations thereof.

29. The method of claim 23, wherein said composition is administered in an amount sufficient to induce said controlled PVD.

30. The method of claim 23, wherein said composition is administered intravitreally.

31. A kit for producing a composition useful for inducing a controlled PVD, the kit comprising:
(a) plasmin or an enzymatically equivalent derivative thereof disposed in a first container; and
(b) at least an anti-inflammatory medicament disposed in a second container, wherein contents of said first and second containers are combined to produce said composition.

32. The kit of claim 31, wherein said at least an anti-inflammatory medicament comprises a corticosteroid, an NSAID, or a PPARγ agonist.

33. The kit of claim 31, wherein said at least an anti-inflammatory medicament is in a form of micrometer-sized solid particles dispersed in a liquid medium.

34. The kit of claim 31, wherein a content of said first container has a pH of less than about 5.

35. The kit of claim 31, wherein the enzymatically equivalent derivative of plasmin is selected from the group consisting of microplasmin, miniplasmin, truncated forms of plasmin, variants of plasmin, combinations thereof, and mixtures thereof.

36. The kit of claim 31, wherein said second container further contains a stabilizing agent for said plasmin or said enzymatically equivalent derivative thereof.
